(19) United States
(12) Patent Application Publication
(10) Pub. No.: US 2011/0172638 A1
(43) Pub. Date: Jul. 14, 2011

(54) DRUG DELIVERY DEVICE INCLUDING MULTI-FUNCTIONAL COVER

(75) Inventors: Benjamin J. Moga, Madison, WI (US); Kent Chase, Sun Prairie, WI (US); Garrick D.S. Smith, Madison, WI (US)

(73) Assignees: Ratio, Inc.; FluGen, Inc.

(21) Appl. No.: 12/684,840

(22) Filed: Jan. 8, 2010

(51) Int. Cl. A61M 5/32

(52) U.S. Cl. 604/506; 604/239

(57) ABSTRACT

An apparatus for delivering a drug to a subject is provided. The apparatus includes a housing, a microneedle coupled to the housing and configured to extend from the housing when activated, an activation control coupled to the housing and an outer shell. The outer shell includes a top wall having an inner surface and a sidewall extending from the top wall, the sidewall having an inner surface. The outer shell includes a first attachment structure configured to attach to the housing. The outer shell covers the activation control when the first attachment structure is attached to the housing. The outer shell includes a second attachment structure configured to attach to the housing. The outer shell covers the activated microneedle when the second attachment structure is attached to the housing.

### BACKGROUND

The present invention relates generally to the field of drug delivery devices. The present invention relates specifically to an active transdermal drug delivery device assembly that uses a microneedle as the point of drug delivery and includes a protective cover.

An active agent or drug (e.g., pharmaceuticals, vaccines, hormones, nutrients, etc.) may be administered to a patient through various means. For example, a drug may be ingested, inhaled, injected, delivered intravenously, etc. In some applications, a drug may be administered transdermally. In some transdermal applications, such as transdermal nicotine or birth control patches, a drug is absorbed through the skin. Passive transdermal patches often include an absorbent layer or membrane that is placed on the outer layer of the skin. The membrane typically contains a dose of a drug that is allowed to be absorbed through the skin to deliver the substance to the patient. Typically, only drugs that are readily absorbed through the outer layer of the skin may be delivered with such devices.

Other drug delivery devices are configured to provide for increased skin permeability to the delivered drugs. For example, some devices use a structure, such as one or more microneedles, to facilitate transfer of the drug into the skin. Solid microneedles may be coated with a dry drug substance. The puncture of the skin by the solid microneedles increases permeability of the skin allowing for absorption of the drug substance. Hollow microneedles may be used to provide a fluid channel for drug delivery below the outer layer of the skin. Other active transdermal devices utilize other mechanisms (e.g., iontophoresis, sonophoresis, etc.) to increase skin permeability to facilitate drug delivery.

### SUMMARY

One embodiment of the invention relates to an apparatus for delivering a drug to a subject. The apparatus includes a housing, a microneedle coupled to the housing and configured to extend from the housing when activated, an activation control coupled to the housing and an outer shell. The outer shell includes a top wall having an inner surface and a sidewall extending from the top wall, the sidewall having an inner surface. The outer shell includes a first attachment structure configured to attach to the housing. The outer shell covers the activation control when the first attachment structure is attached to the housing. The outer shell includes a second attachment structure configured to attach to the housing. The outer shell covers the activated microneedle when the second attachment structure is attached to the housing.

Another embodiment of the invention relates to an apparatus for delivering drug to a subject. The apparatus includes a housing, a microneedle configured to extend from the housing when activated, an activation control coupled to the housing and an outer shell coupled to the housing. The outer shell includes a top wall having an inner surface and a sidewall extending from a peripheral edge of the top wall. The sidewall includes an inner surface, and the inner surfaces of the top wall and the sidewall define a central chamber. The outer shell includes a first attachment structure coupled to the housing. The housing and the activation control are located within the central chamber when the outer shell is coupled to the housing via the first attachment structure. The outer shell includes a second attachment structure configured to be coupled to the housing. The activated microneedle is located within the central chamber when the outer shell is coupled to the housing via the second attachment structure.

Another embodiment of the invention relates to a method of delivering a drug to the skin of a subject. The method includes providing a microneedle drug delivery device held within a protective cover and attaching the microneedle drug delivery device to the skin of the subject via an attachment element. The method includes removing the protective cover from the microneedle drug delivery device while the microneedle drug delivery device is attached to the skin of the subject to expose an activation control and actuating the activation control to trigger insertion of a microneedle into the skin of the subject and to initiate drug delivery via the microneedle. The method includes removing the microneedle drug delivery device from the skin of the subject and attaching the microneedle drug delivery device to the protective cover for disposal such that the exposed microneedle is covered by the protective cover.

Alternative exemplary embodiments relate to other features and combinations of features as may be generally recited in the claims

### BRIEF DESCRIPTION OF THE FIGURES

This application will become more fully understood from the following detailed description, taken in conjunction with the accompanying figures, wherein like reference numerals refer to like elements in which:

FIG. 1 is a perspective view of a drug delivery device assembly having a cover and a protective membrane according to an exemplary embodiment;

FIG. 2 is a perspective view of a drug delivery device according to an exemplary embodiment after both the cover and protective membrane have been removed;

FIG. 3 is a exploded perspective view of a drug delivery device assembly according to an exemplary embodiment;

FIG. 4 is a exploded perspective view of a drug delivery device showing various components mounted within the device housing according to an exemplary embodiment;

FIG. 5 is a exploded perspective view of a drug delivery device showing various components removed from the device housing according to an exemplary embodiment;

FIG. 6 is a perspective sectional view showing a drug delivery device prior to activation according to an exemplary embodiment;

FIG. 7 is a perspective sectional view showing a drug delivery device following activation according to an exemplary embodiment;

FIG. 8 is a side sectional view showing a drug delivery device following activation according to an exemplary embodiment;

FIG. 9 is a side sectional view showing a drug delivery device following delivery of a drug according to an exemplary embodiment;

FIG. 10 is a perspective view of a drug delivery device assembly having a cover and a protective membrane according to an exemplary embodiment;

FIG. 11 is a side sectional view showing a drug delivery device assembly according to an exemplary embodiment;

FIG. 12 is a perspective view of a drug delivery device assembly prior to attachment of the drug delivery device to the skin of a subject;

FIG. 13 is a perspective view of a drug delivery device assembly after attachment of the drug delivery device to the skin of a subject;

FIG. 14 is a perspective view of a drug delivery device assembly after attachment of the drug delivery device to the skin of a subject and after removal of a protective cover;

FIG. 15 is side sectional view showing a drug delivery device assembly prepared for disposal according to an exemplary embodiment; and

FIG. 16 is an enlarged view showing engagement between a protective cover and a drug delivery device prepared for disposal according to an exemplary embodiment.

### DETAILED DESCRIPTION

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring generally to the figures, a substance delivery device assembly is shown according to various exemplary embodiments. The delivery device assembly includes various packaging and/or protective elements that provide for protection during storage and transportation. The assembly also includes a substance delivery device that is placed in contact with the skin of a subject (e.g., a human or animal, etc.) prior to delivery of the substance to the subject. After the device is affixed to the skin of the subject, the device is activated in order to deliver the substance to the subject. Following delivery of the substance, the device is removed from the skin.

The delivery device described herein may be utilized to deliver any substance that may be desired. In one embodiment, the substance to be delivered is a drug, and the delivery device is a drug delivery device configured to deliver the drug to a subject. As used herein the term “drug” is intended to include any substance delivered to a subject for any therapeutic, preventative or medicinal purpose (e.g., vaccines, pharmaceuticals, nutrients, nutraceuticals, etc.). In one such embodiment, the drug delivery device is a vaccine delivery device configured to deliver a dose of vaccine to a subject. In one embodiment, the delivery device is configured to deliver a flu vaccine. The embodiments discussed herein relate primarily to a device configured to deliver a substance intradermally. In other embodiments, the device may be configured to deliver a substance transdermally or may be configured to deliver drugs directly to an organ other than the skin.

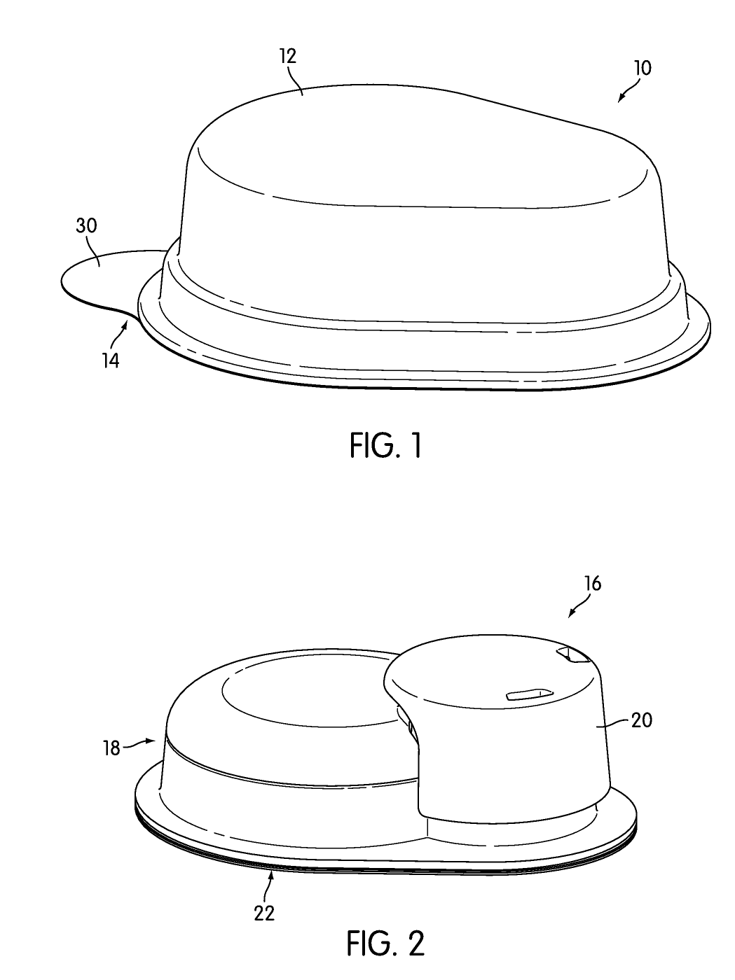

Referring to FIG. 1, drug delivery device assembly 10 is depicted according to an exemplary embodiment. Drug delivery device assembly 10 includes an outer protective cover 12 and a protective membrane or barrier 14 that provides a sterile seal for drug delivery device assembly 10. As shown in FIG. 1, drug delivery device assembly 10 is shown with cover 12 and protective barrier 14 in an assembled configuration. Generally, cover 12 and protective barrier 14 protect various components of drug delivery device 16 during storage and transport prior to use by the end user. In various embodiments, cover 12 may be made of a relatively rigid material (e.g., plastic, metal, cardboard, etc.) suitable to protect other components of drug delivery device assembly 10 during storage or shipment. As shown, cover 12 is made from a non-transparent material. However, in other embodiments cover 12 is a transparent or semi-transparent material.

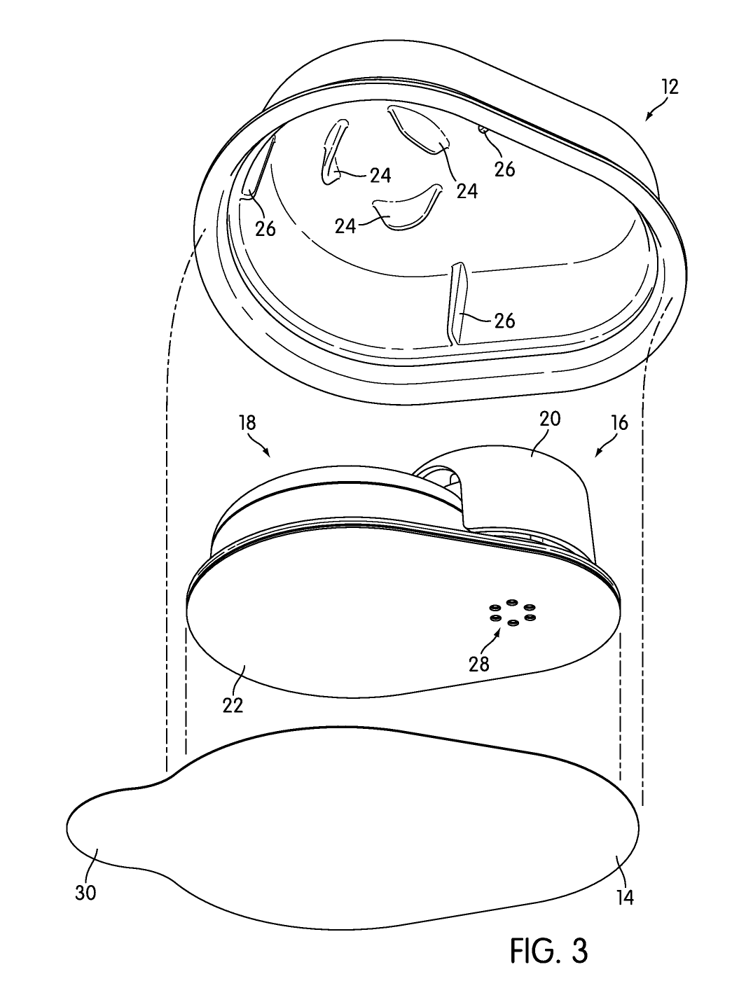

As shown in FIG. 2 and FIG. 3, the drug delivery device assembly includes delivery device 16. Delivery device 16 includes a housing 18, an activation control, shown as, but not limited to, button 20, and an attachment element, shown as, but not limited to, adhesive layer 22. Adhesive layer 22 includes one or more holes 28 (see FIG. 3). Holes 28 provide a passageway for one or more hollow drug delivery microneedles as discussed in more detail below. During storage and transport, cover 12 is mounted to housing 18 of delivery device 16 such that delivery device 16 is received within cover 12. In the embodiment shown, cover 12 includes three projections or tabs 24 extending from the inner surface of the top wall of cover 12 and three projections or tabs 26 extending from the inner surface of the sidewall of cover 12. When cover 12 is mounted to delivery device 16, tabs 24 and 26 contact the outer surface of housing 18 such that delivery device 16 is positioned properly and held within cover 12. Protective barrier 14 is attached to the lower portion of cover 12 covering adhesive layer 22 and holes 28 during storage and shipment. Together, cover 12 and protective barrier 14 act to provide a sterile and hermetically sealed packaging for delivery device 16.

Referring to FIG. 3, to use delivery device 16 to deliver a drug to a subject, protective barrier 14 is removed exposing adhesive layer 22. In the embodiment shown, protective barrier 14 includes a tab 30 that facilitates griping of protective barrier 14 during removal. Once adhesive layer 22 is exposed, delivery device 16 is placed on the skin. Adhesive layer 22 is made from an adhesive material that forms a nonpermanent bond with the skin of sufficient strength to hold delivery device 16 in place on the skin of the subject during use. Cover 12 is released from delivery device 16 exposing housing 18 and button 20 by squeezing the sides of cover 12. With delivery device 16 adhered to the skin of the subject, button 20 is pressed to trigger delivery of the drug to the patient. When delivery of the drug is complete, delivery device 16 may be detached from the skin of the subject by applying sufficient force to overcome the grip generated by adhesive layer 22.

In one embodiment, delivery device 16 is sized to be conveniently wearable by the user during drug delivery. In one embodiment, the length of delivery device 16 along the device's long axis is 53.3 mm, the length of delivery device 16 along the device's short axis (at its widest dimension) is 48 mm, and the height of delivery device 16 at button 20 following activation is 14.7 mm. However, in other embodiments other dimensions are suitable for a wearable drug delivery device. For example, in another embodiment, the length of delivery device 16 along the device's long axis is between 40 mm and 80 mm, the length of delivery device 16 along the device's short axis (at its widest dimension) is between 30 mm and 60 mm, and the height of delivery device 16 at button 20 following activation is between 5 mm and 30 mm. In another embodiment, the length of delivery device 16 along the device's long axis is between 50 mm and 55 mm, the length of delivery device 16 along the device's short axis (at its widest dimension) is between 45 mm and 50 mm, and the height of delivery device 16 at button 20 following activation is between 10 mm and 20 mm.

While in the embodiments shown the attachment element is shown as, but not limited to, adhesive layer 22, other attachment elements may be used. For example, in one embodiment, delivery device 16 may be attached via an elastic strap. In another embodiment, delivery device 16 may not include an attachment element and may be manually held in place during delivery of the drug. Further, while the activation control is shown as button 20, the activation control may be a switch, trigger, or other similar element, or may be more than one button, switch, trigger, etc., that allows the user to trigger delivery of the drug.

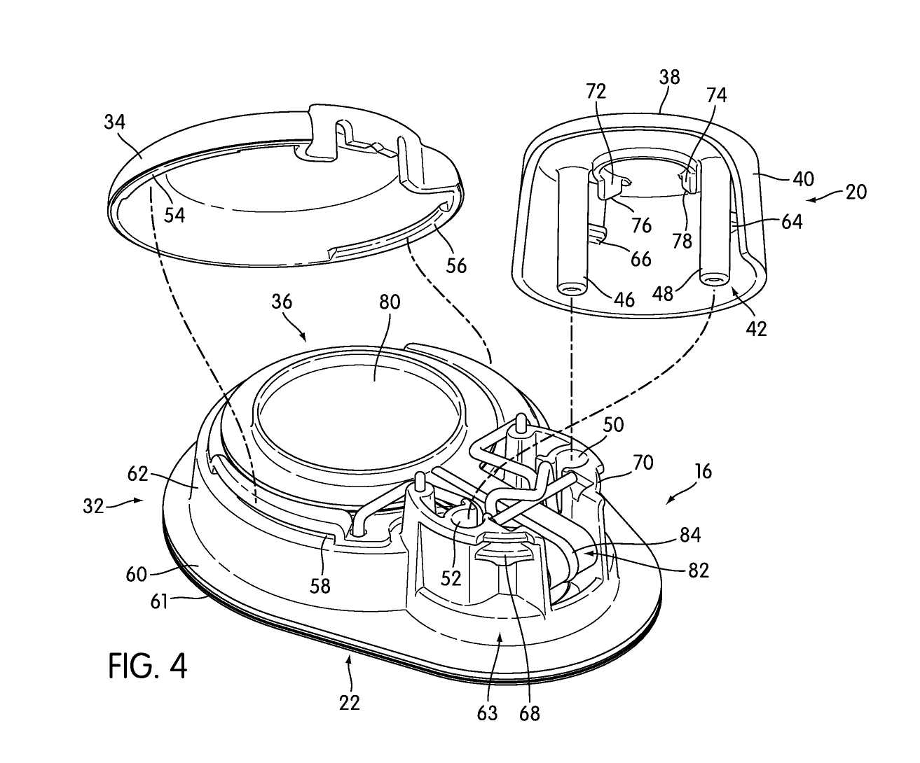

Referring to FIG. 4, housing 18 of delivery device 16 includes a base portion 32 and a reservoir cover 34. Base portion 32 includes a flange 60, a bottom tensile member, shown as bottom wall 61, a first support portion 62 and a second support portion 63. In the embodiment shown, bottom wall 61 is a rigid wall that is positioned below flange 60. As shown in FIG. 4, the outer surface of first support portion 62 is generally cylindrically shaped and extends upward from flange 60. Second support portion 63 is generally cylindrically shaped and extends upward from flange 60 to a height above first support portion 62. As shown in FIG. 4, delivery device 16 includes a substance delivery assembly 36 mounted within base portion 32 of housing 18.

Reservoir cover 34 includes a pair of tabs 54 and 56 that each extend inwardly from a portion of the inner edge of cover 34. Base portion 32 includes a recess 58 and second recess similar to recess 58 on the opposite side of base portion 32. As shown in FIG. 4, both recess 58 and the opposing recess are formed in the upper peripheral edge of the outer surface of first support portion 62. When reservoir cover 34 is mounted to base portion 32, tab 54 is received within recess 58 and tab 56 is received within the similar recess on the other side of base portion 32 to hold cover 34 to base portion 32.

As shown in FIG. 4, button 20 includes a top wall 38. Button 20 also includes a sidewall or skirt 40 that extends from a portion of the peripheral edge of top wall 38 such that skirt 40 defines an open segment 42. Button 20 is shaped to receive the generally cylindrical shaped second support portion 63 of base portion 32. Button 20 includes a first mounting post 46 and a second mounting post 48 both extending in a generally perpendicular direction from the lower surface of top wall 38. Second support portion 63 includes a first channel 50 and a second channel 52. Mounting posts 46 and 48 are slidably received within channels 50 and 52, respectively, when button 20 is mounted to second support portion 63. Mounting posts 46 and 48 and channels 50 and 52 act as a vertical movement guide for button 20 to help ensure that button 20 moves in a generally downward vertical direction in response to a downward force applied to top wall 38 during activation of delivery device 16. Precise downward movement of button 20 ensures button 20 interacts as intended with the necessary components of substance delivery assembly 36 during activation.

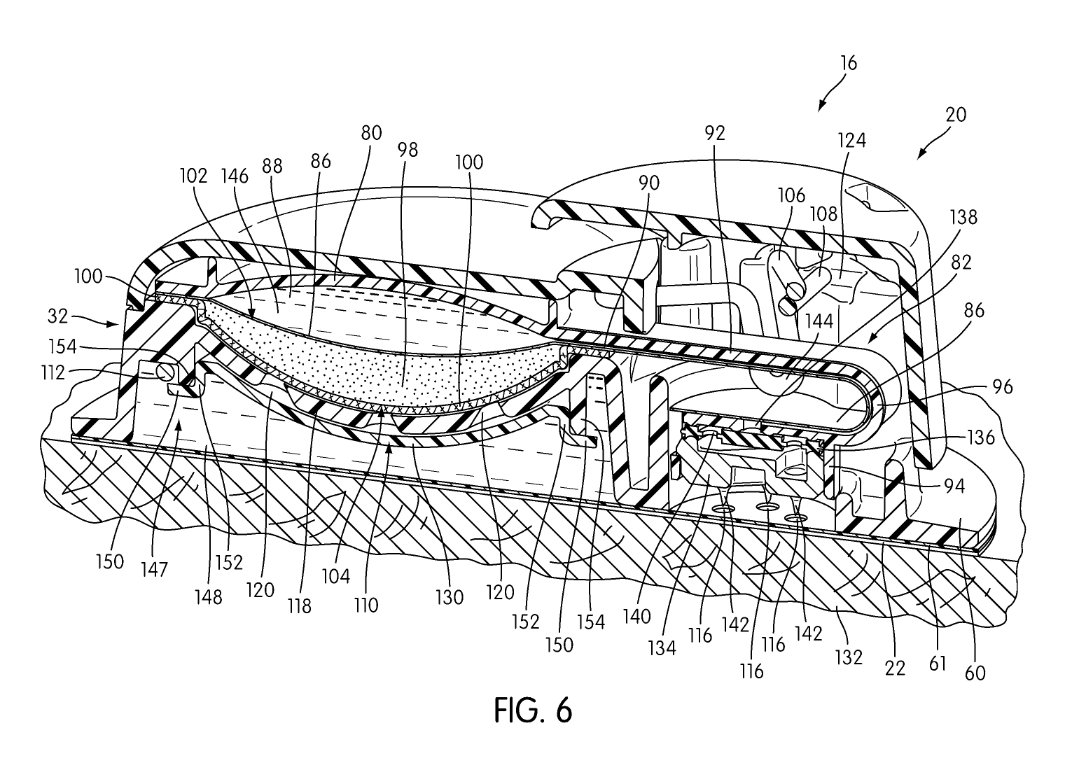

Button 20 also includes a first support ledge 64 and a second support ledge 66 both extending generally perpendicular to the inner surface of sidewall 40. The outer surface of second support portion 63 includes a first button support surface 68 and second button support surface 70. When button 20 is mounted to second support portion 63, first support ledge 64 engages and is supported by first button support surface 68 and second support ledge 66 engages and is supported by second button support surface 70. The engagement between ledge 64 and surface 68 and between ledge 66 and surface 70 supports button 20 in the pre-activation position (shown for example in FIG. 6). Button 20 also includes a first latch engagement element 72 and a second latch engagement element 74 both extending in a generally perpendicular direction from the lower surface of top wall 38. First latch engagement element 72 includes an angled engagement surface 76 and second latch engagement element 74 includes an angled engagement surface 78.

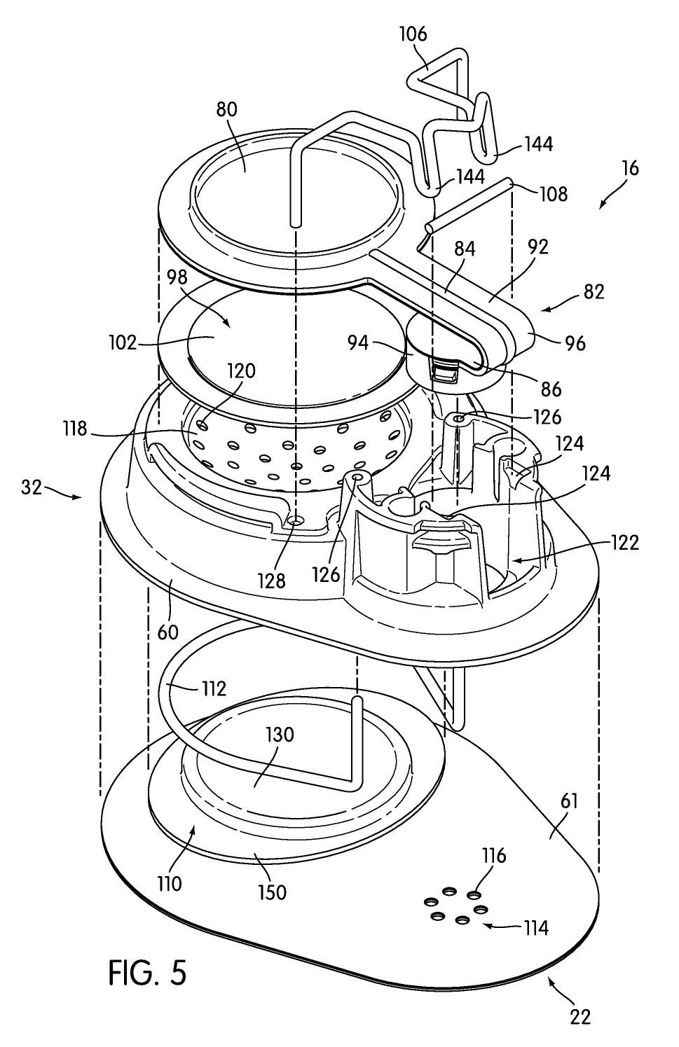

Referring to FIG. 4 and FIG. 5, substance delivery assembly 36 includes a drug reservoir base 80 and drug channel arm 82. The lower surface of drug channel arm 82 includes a depression or groove 84 that extends from reservoir base 80 along the length of drug channel arm 82. As shown in FIG. 4 and FIG. 5, groove 84 appears as a rib protruding from the upper surface of drug channel arm 82. Substance delivery assembly 36 further includes a flexible barrier film 86 adhered to the inner surfaces of both drug reservoir base 80 and drug channel arm 82. Barrier film 86 is adhered to form a fluid tight seal or a hermetic seal with drug reservoir base 80 and channel arm 82. In this arrangement (shown best in FIGS. 6-9), the inner surface of drug reservoir base 80 and the inner surface of barrier film 86 form a drug reservoir 88, and the inner surface of groove 84 and the inner surface of barrier film 86 form a fluid channel, shown as, but not limited to, drug channel 90. In this embodiment, drug channel arm 82 acts as a conduit to allow fluid to flow from drug reservoir 88. As shown, drug channel arm 82 includes a first portion 92 extending from drug reservoir base 80, a microneedle attachment portion, shown as, but not limited to, cup portion 94, and a generally U-shaped portion 96 joining the first portion 92 to the cup portion 94. In the embodiment shown, drug reservoir base 80 and drug channel arm 82 are made from an integral piece of polypropylene. However, in other embodiments, drug reservoir base 80 and drug channel arm 82 may be separate pieces joined together and may be made from other plastics or other materials.

Substance delivery assembly 36 includes a reservoir actuator or force generating element, shown as, but not limited to, hydrogel 98, and a fluid distribution element, shown as, but not limited to, wick 100 in FIG. 6. Because FIG. 5 depicts delivery device 16 in the pre-activated position, hydrogel 98 is formed as a hydrogel disc and includes a concave upper surface 102 and a convex lower surface 104. As shown, wick 100 is positioned below hydrogel 98 and is shaped to generally conform to the convex shape of lower surface 104.

Substance delivery assembly 36 includes a microneedle activation element or microneedle actuator, shown as, but not limited to, torsion rod 106, and a latch element, shown as, but not limited to, latch bar 108. As explained in greater detail below, torsion rod 106 stores energy, which upon activation of delivery device 16, is transferred to one or more microneedles causing the microneedles to penetrate the skin. Substance delivery assembly 36 also includes a fluid reservoir plug 110 and plug disengagement bar 112. Bottom wall 61 is shown removed from base portion 32, and adhesive layer 22 is shown coupled to the lower surface of bottom wall 61. Bottom wall 61 includes one or more holes 114 that are sized and positioned to align with holes 28 in adhesive layer 22. In this manner, holes 114 in bottom wall 61 and holes 28 in adhesive layer 22 form channels, shown as needle channels 116.

As shown in FIG. 5, first support portion 62 includes a support wall 118 that includes a plurality of fluid channels 120. When assembled, wick 100 and hydrogel 98 are positioned on support wall 118 below drug reservoir 88. As shown, support wall 118 includes an upper concave surface that generally conforms to the convex lower surfaces of wick 100 and hydrogel 98. Fluid reservoir plug 110 includes a concave central portion 130 that is shaped to generally conform to the convex lower surface of support wall 118. First support portion 62 also includes a pair of channels 128 that receive the downwardly extending segments of torsion rod 106 such that the downwardly extending segments of torsion rod 106 bear against the upper surface of bottom wall 61 when delivery device 16 is assembled. Second support portion 63 includes a central cavity 122 that receives cup portion 94, U-shaped portion 96 and a portion of first portion 92 of drug channel arm 82. Second support portion 63 also includes a pair of horizontal support surfaces 124 that support latch bar 108 and a pair of channels 126 that slidably receive the vertically oriented portions of plug disengagement bar 112.

Referring to FIG. 6, a perspective, sectional view of delivery device 16 is shown attached or adhered to skin 132 of a subject prior to activation of the device. As shown, adhesive layer 22 provides for gross attachment of the device to skin 132 of the subject. Delivery device 16 includes a microneedle component, shown as, but not limited to, microneedle array 134, having a plurality of microneedles, shown as, but not limited to, hollow microneedles 142, extending from the lower surface of microneedle array 134. In the embodiment shown, microneedle array 134 includes an internal channel 141 allowing fluid communication from the upper surface of microneedle array 134 to the tips of hollow microneedles 142. Delivery device 16 also includes a valve component, shown as, but not limited to, check valve 136. Both microneedle array 134 and check valve 136 are mounted within cup portion 94. Drug channel 90 terminates in an aperture or hole 138 positioned above check valve 136. In the pre-activation or inactive position shown in FIG. 6, check valve 136 blocks hole 138 at the end of drug channel 90 preventing a substance, shown as, but not limited to, drug 146, within drug reservoir 88 from flowing into microneedle array 134. While the embodiments discussed herein relate to a drug delivery device that utilizes hollow microneedles, in other various embodiments, other microneedles, such as solid microneedles, may be utilized.

As shown in FIG. 6, in the pre-activation position, latch bar 108 is supported by horizontal support surfaces 124. Latch bar 108 in turn supports torsion rod 106 and holds torsion rod 106 in the torqued, energy storage position shown in FIG. 6. Torsion rod 106 includes a U-shaped contact portion 144 that bears against a portion of the upper surface of barrier film 86 located above cup portion 94. In another embodiment, U-shaped contact portion 144 is spaced above barrier film 86 (i.e., not in contact with barrier film 86) in the pre-activated position.

Delivery device 16 includes an activation fluid reservoir, shown as, but not limited to, fluid reservoir 147, that contains an activation fluid, shown as, but not limited to, water 148. In the embodiment shown, fluid reservoir 147 is positioned generally below hydrogel 98. In the pre-activation position of FIG. 6, fluid reservoir plug 110 acts as a plug to prevent water 148 from flowing from fluid reservoir 147 to hydrogel 98. In the embodiment show, reservoir plug 110 includes a generally horizontally positioned flange 150 that extends around the periphery of plug 110. Reservoir plug 110 also includes a sealing segment 152 that extends generally perpendicular to and vertically away from flange 150. Sealing segment 152 of plug 110 extends between and joins flange 150 with the concave central portion 130 of plug 110. The inner surface of base portion 32 includes a downwardly extending annular sealing segment 154. The outer surfaces of sealing segment 152 and/or a portion of flange 150 abut or engage the inner surface of annular sealing segment 154 to form a fluid-tight seal preventing water from flowing from fluid reservoir 147 to hydrogel 98 prior to device activation.

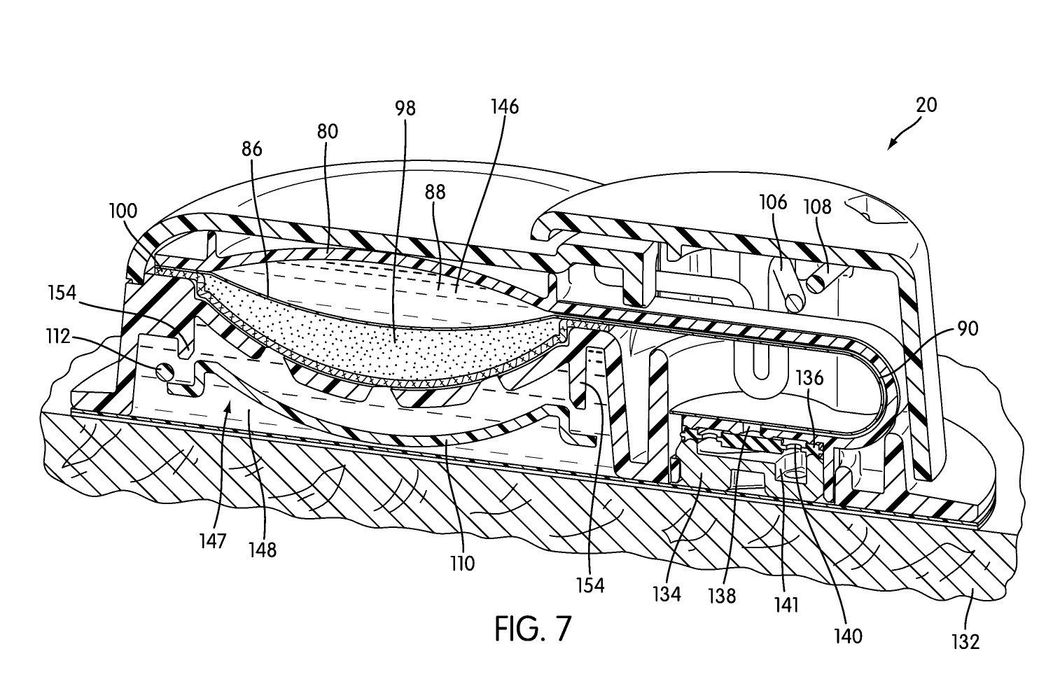

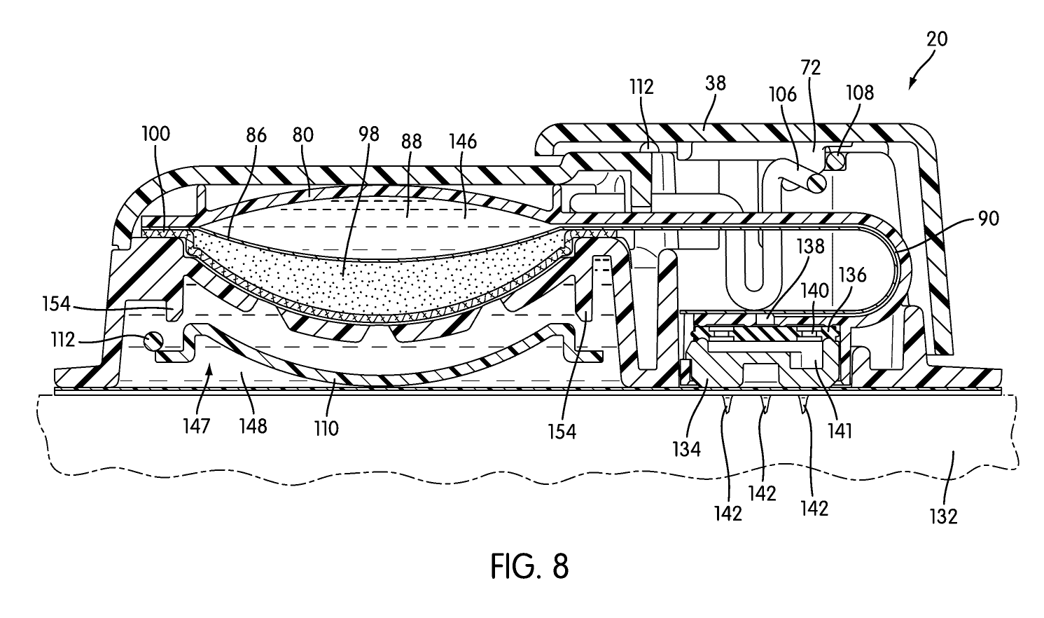

Referring to FIG. 7 and FIG. 8, delivery device 16 is shown immediately following activation. In FIG. 8, skin 132 is drawn in broken lines to show hollow microneedles 142 after insertion into the skin of the subject. To activate delivery device 16, button 20 is pressed in a downward direction (toward the skin). Movement of button 20 from the pre-activation position of FIG. 6 to the activated position causes activation of both microneedle array 134 and of hydrogel 98. Depressing button 20 causes first latch engagement element 72 and second latch engagement element 74 to engage latch bar 108 and to force latch bar 108 to move from beneath torsion rod 106 allowing torsion rod 106 to rotate from the torqued position of FIG. 6 to the seated position of FIG. 7. The rotation of torsion rod 106 drives microneedle array 134 downward and causes hollow microneedles 142 to pierce skin 132. In addition, depressing button 20 causes the lower surface of button top wall 38 to engage plug disengagement bar 112 forcing plug disengagement bar 112 to move downward. As plug disengagement bar 112 is moved downward, fluid reservoir plug 110 is moved downward breaking the seal between annular sealing segment 154 of base portion 32 and sealing segment 152 of reservoir plug 110.

With the seal broken, water 148 within reservoir 147 is put into fluid communication with hydrogel 98. As water 148 is absorbed by hydrogel 98, hydrogel 98 expands pushing barrier film 86 upward toward drug reservoir base 80. As barrier film 86 is pushed upward by the expansion of hydrogel 98, pressure within drug reservoir 88 and drug channel 90 increases. When the fluid pressure within drug reservoir 88 and drug channel 90 reaches a threshold, check valve 136 is forced open allowing drug 146 within drug reservoir 88 to flow through aperture 138 at the end of drug channel 90. As shown, check valve 136 includes a plurality of holes 140, and microneedle array 134 includes a plurality of hollow microneedles 142. Drug channel 90, hole 138, plurality of holes 140 of check valve 136, internal channel 141 of microneedle array 134 and hollow microneedles 142 define a fluid channel between drug reservoir 88 and the subject when check valve 136 is opened. Thus, drug 146 is delivered from reservoir 88 through drug channel 90 and out of the holes in the tips of hollow microneedles 142 to the skin of the subject by the pressure generated by the expansion of hydrogel 98.

In the embodiment shown, check valve 136 is a segment of flexible material (e.g., medical grade silicon) that flexes away from aperture 138 when the fluid pressure within drug channel 90 reaches a threshold placing drug channel 90 in fluid communication with hollow microneedles 142. In one embodiment, the pressure threshold needed to open check valve 136 is about 0.5-1.0 pounds per squire inch (psi). In various other embodiments, check valve 136 may be a rupture valve, a swing check valve, a ball check valve, or other type of valve the allows fluid to flow in one direction. In the embodiment shown, the microneedle actuator is a torsion rod 106 that stores energy for activation of the microneedle array until the activation control, shown as button 20, is pressed. In other embodiments, other energy storage or force generating components may be used to activate the microneedle component. For example, in various embodiments, the microneedle activation element may be a coiled compression spring or a leaf spring. In other embodiments, the microneedle component may be activated by a piston moved by compressed air or fluid. Further, in yet another embodiment, the microneedle activation element may be an electromechanical element, such as a motor, operative to push the microneedle component into the skin of the patient.

In the embodiment shown, the actuator that provides the pumping action for drug 146 is a hydrogel 98 that expands when allowed to absorb water 148. In other embodiments, hydrogel 98 may be an expandable substance that expands in response to other substances or to changes in condition (e.g., heating, cooling, pH, etc.). Further, the particular type of hydrogel utilized may be selected to control the delivery parameters. In various other embodiments, the actuator may be any other component suitable for generating pressure within a drug reservoir to pump a drug in the skin of a subject. In one exemplary embodiment, the actuator may be a spring or plurality of springs that when released push on barrier film 86 to generate the pumping action. In another embodiment, the actuator may be a manual pump (i.e., a user manually applies a force to generate the pumping action). In yet another embodiment, the actuator may be an electronic pump.

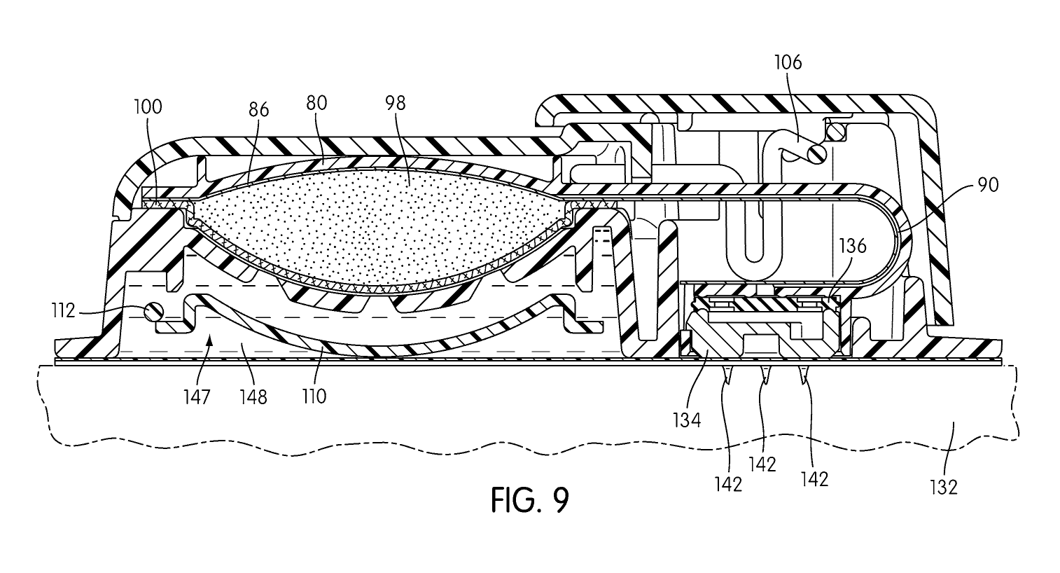

Referring to FIG. 9, delivery device 16 is shown following completion of delivery of drug 146 to the subject. In FIG. 9, skin 132 is drawn in broken lines. As shown in FIG. 9, hydrogel 98 expands until barrier film 86 is pressed against the lower surface of reservoir base 80. When hydrogel 98 has completed expansion, substantially all of drug 146 has been pushed from drug reservoir 88 into drug channel 90 and delivered to skin 132 of the subject. The volume of drug 146 remaining within delivery device 16 (i.e., the dead volume) following complete expansion by hydrogel 98 is minimized by configuring the shape of drug reservoir 88 to enable complete evacuation of the drug reservoir and by minimizing the volume of fluid pathway formed by drug channel 90, hole 138, plurality of holes 140 of check valve 136 and hollow microneedles 142. In the embodiment shown, delivery device 16 is a single-use, disposable device that is detached from skin 132 of the subject and is discarded when drug delivery is complete. However, in other embodiments, delivery device 16 may be reusable and is configured to be refilled with new drug, to have the hydrogel replaced, and/or to have the microneedles replaced.

In one embodiment, delivery device 16 and reservoir 88 are sized to deliver a dose of drug of up to approximately 500 microliters. In other embodiments, delivery device 16 and reservoir 88 are sized to allow delivery of other volumes of drug (e.g., up to 200 microliters, up to 400 microliters, up to 1 milliliter, etc.).

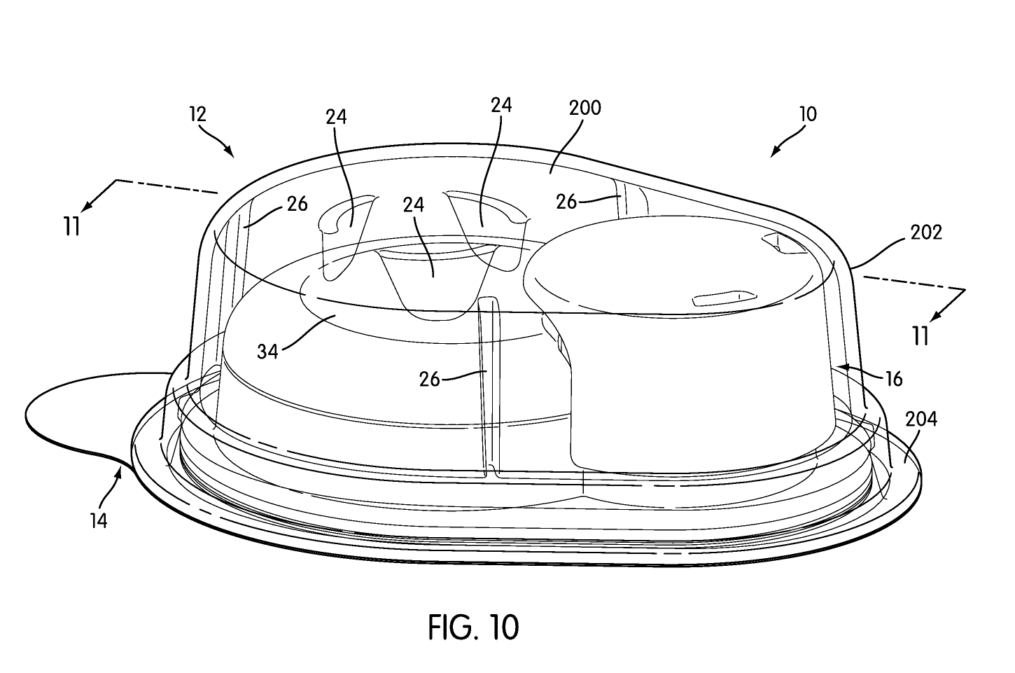

Referring generally to FIGS. 10-16, various embodiments of a substance delivery device assembly including a protective shell are shown. FIG. 10 shows a perspective view of drug delivery device assembly 10 in the assembled configuration for transport or storage. As discussed above, delivery device assembly 10 includes an outer shell or case, shown as cover 12, and a protective barrier 14. Protective barrier 14 is attached to cover 12 such that drug delivery device 16 is sealed within a chamber formed by the upper surface of protective barrier 14 and the inner surface of cover 12. In one embodiment, cover 12 may be made from a transparent or translucent material (see FIG. 10), and in another embodiment, cover 12 may be made from a nontransparent material.

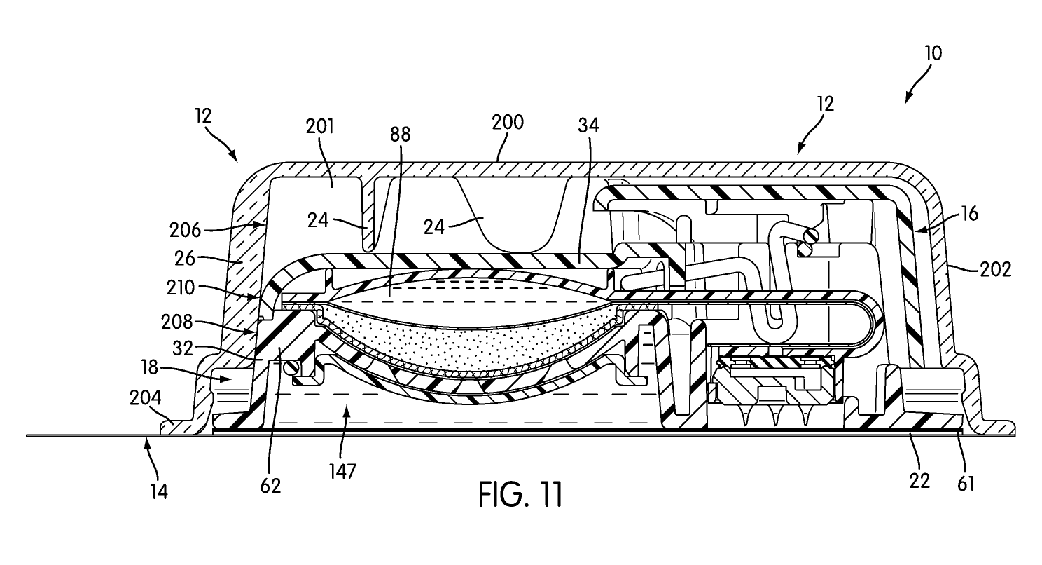

As shown in FIGS. 10 and 11, cover 12 includes a top wall 200 and a sidewall 202 extending from the peripheral edge of top wall 200. In the embodiment shown, top wall 200 is a generally planar structure. In other embodiments, cover 12 is generally domed-shaped with top wall 200 being an outwardly curved surface. Cover 12 includes a central chamber 201 that is defined by the inner surfaces of top wall 200 and sidewall 202. As shown, in the assembled configuration, delivery device 16, including housing 18 and button 20, are located within central chamber 201.

Extending outwardly from the lower, peripheral edge of sidewall 202 is a flange 204. With delivery device 16 positioned within cover 12, protective barrier 14 is adhered to the lower surface of flange 204 to form delivery device assembly 10. In one embodiment, the seal formed between protective barrier 14 and flange 204 is a hermetic seal. In this embodiment, the hermetic seal between protective barrier 14 and flange 204 provides a sterile barrier to ensure that delivery device 16 remains sterile within delivery device assembly 10. Further, in one embodiment, both cover 12 and protective barrier 14 are both made from rigid materials to provide protection for delivery device 16 during transportation and storage. Further, rigidity of cover 12 and of protective barrier 14 acts to resist or prevent deformation due to changes in air pressure (e.g., during air transport) that may otherwise create a device malfunction or that may compromise device safety and/or efficacy.

In addition to providing a sterile seal, the hermetic seal formed between protective barrier 14 and flange 204 provides for a low evaporation rate for the various liquids contained within delivery device 16. The hermetic seal lowers the evaporation rate for the activation fluid (e.g., water) within fluid reservoir 147 such that sufficient activation fluid is within fluid reservoir 147 to provide the force necessary for drug delivery at the time of use. The hermetic seal also lowers the evaporation rate of the liquid drug within drug reservoir 88 such that the concentration of liquid drug remains within a suitable range at the time of use. Because the seal between protective barrier 14 and flange 204 lowers evaporation rate, the seal acts to increase the shelf-life of delivery device assembly 10.

Cover 12 includes various structures to provide support for and attachment to delivery device 16 when cover 12 is attached to delivery device 16. Cover 12 includes three tabs 24 extending from the lower surface of top wall 200. When cover 12 is attached to delivery device 16, tabs 24 contact the upper surface of reservoir cover 34. The contact between tabs 24 and upper surface of reservoir cover 34 provides support for delivery device 16 and limits vertical movement of delivery device 16 within cover 12.

Cover 12 includes a first or device attachment structure, shown as tabs 26 in FIG. 10, configured to engage housing 18 of delivery device 16 in the assembled configuration. In the assembled configuration, the housing of delivery device 16 and button 20 are received within central chamber 201 of cover 12 such that cover 12 covers (e.g., conceals, envelopes, houses, etc.) the housing of delivery device 16 and button 20. Tabs 26 are also shown in the perspective view of FIG. 3. Tabs 26 extend outwardly from the inner surface of sidewall 202 generally toward the interior of cover 12. In the vertical direction, tabs 26 extend from the lower surface of top wall 200 along the inner surface of sidewall 200 toward the lower edge of cover 12. In the embodiment shown, tabs 26 extend approximately seventy percent of the distance from top wall 200 to cover 12.

Referring to FIG. 11, tabs 26 each include an inner surface 206 having a portion configured to engage the outer surface of housing 18 to hold delivery device 16 within cover 12 even following removal of protective barrier 14. As shown in FIG. 11, a portion 208 of the inner surface 206 engages the outer surface of first support portion 62 of base portion 32 of housing 18. In the embodiment shown, a portion 210 of the inner surface 206 engages the outer surface of reservoir cover 34. The engagement between the inner surfaces 206 of tabs 26 acts to attach cover 12 to delivery device 16. In the embodiment shown, tabs 26 form an interference fit with the outer surfaces of first support portion 62 and reservoir cover 34 such that the interference fit supports the weight of delivery device 16 to hold delivery device 16 within cover 12 after protective barrier 14 is removed. It should be understood that while FIG. 11 shows only one of the tabs 26 in engagement with the outer surfaces of first support portion 62 and reservoir cover 34, the other two of the tabs 26 are configured in a similar manner.

While in the embodiments shown, the device attachment structure of cover 12 is depicted as tabs 26 that form a press fit with portions of the outer surface of housing 18, it should be understood that cover 12 may include other device attachment structures. In one embodiment, the outer surface of housing 18 may include one or more slots or recesses that receive one or more tabs extending from the inner surface of cover 12. In another embodiment, cover 12 may include a bead extending along at least a portion of the inner surface of sidewall 202 that is received within a corresponding recess formed in the outer surface of housing 18. In another embodiment, cover 12 may include a recess extending along at least a portion of the inner surface of sidewall 202 that receives a corresponding bead formed in the outer surface of housing 18. In another embodiment, cover 12 may be coupled to housing 18 via a frangible component (e.g., a perforated or weakened strip of material, etc.) that is broken or removed to release delivery device 16 from cover 12.

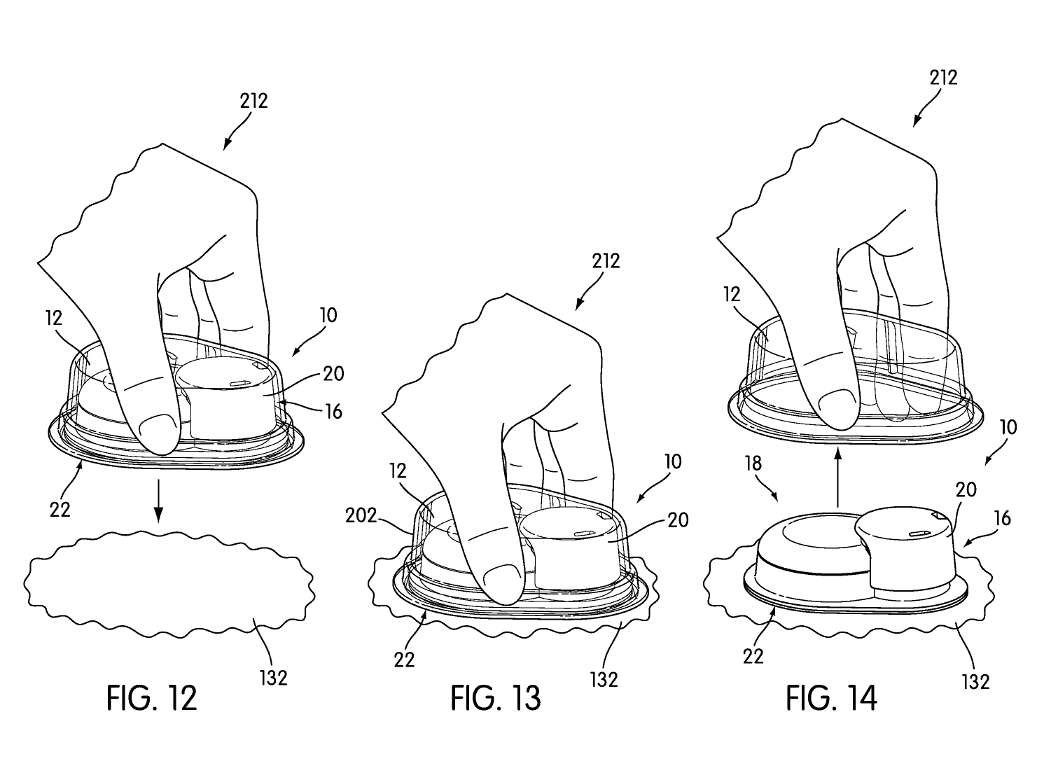

Referring to FIGS. 12-14, attachment of delivery device assembly 10 to skin 132 of a subject is shown according to an exemplary embodiment. In the embodiment shown in FIGS. 12-14, cover 12 functions as a handle or grip that facilitates handling of delivery device 16 by the user 212. Cover 12 facilitates handling by providing a convenient and comfortable grasping surface, by preventing inadvertent contact between adhesive layer 22 and user 212, preventing inadvertent contact between user 212 and button 20, etc. As shown in FIG. 12, following removal of protective barrier 14, cover 12 is grasped by user 212, and delivery device assembly 10 is moved toward skin 132 of the subject with adhesive layer 22 facing skin 132. The interference fit between tabs 26 of cover 12 and housing 18 of delivery device 16, as discussed above and shown in FIG. 10, retains delivery device 16 within cover 12 as user 212 brings delivery device assembly 10 toward skin 132.

As shown in FIG. 13, delivery device assembly 10 is moved downward (toward the subject) such that adhesive layer 22 is brought into contact with skin 132 of the subject. In this position, adhesive layer 22 forms a nonpermanent bond with skin 132 to attach delivery device 16 to skin 132. With adhesive layer 22 attached to skin 132, user 212 may then disengage cover 12 from delivery device 16. In the embodiment shown, to disengage cover 12 from delivery device 16 user 212 squeezes (i.e., applies an inwardly directed force to) the outer surface of sidewall 202 of cover 12. The application of force causes slight deformation of sidewall 202 of cover 12, causing disengagement of one or more of tabs 26 such that cover 12 may be removed from delivery device 16. In other embodiments, cover 12 may be disengaged from delivery device 16 via other mechanisms. For example, in one embodiment, the bond between adhesive layer 22 and skin 132 may be stronger than the interference fit between cover 12 and delivery device 16 such that pulling upwardly on cover 12 will cause disengagement from delivery device 16 without causing adhesive layer 22 to disengage from skin 132. In other embodiments, cover 12 may be disengaged from delivery device 16 via a mechanical latch or button, or via an electronic disengagement mechanism.

As shown in FIG. 14, following disengagement of tabs 26 from delivery device 16, cover 12 is moved upwardly away from skin 132 exposing delivery device 16. Because of the nonpermanent bond between adhesive layer 22 and skin 132, delivery device 16 remains affixed to skin 132 as cover 12 is moved upward. With delivery device 16 attached to skin 132, the drug may be delivered to the subject by pressing button 20, as discussed above.

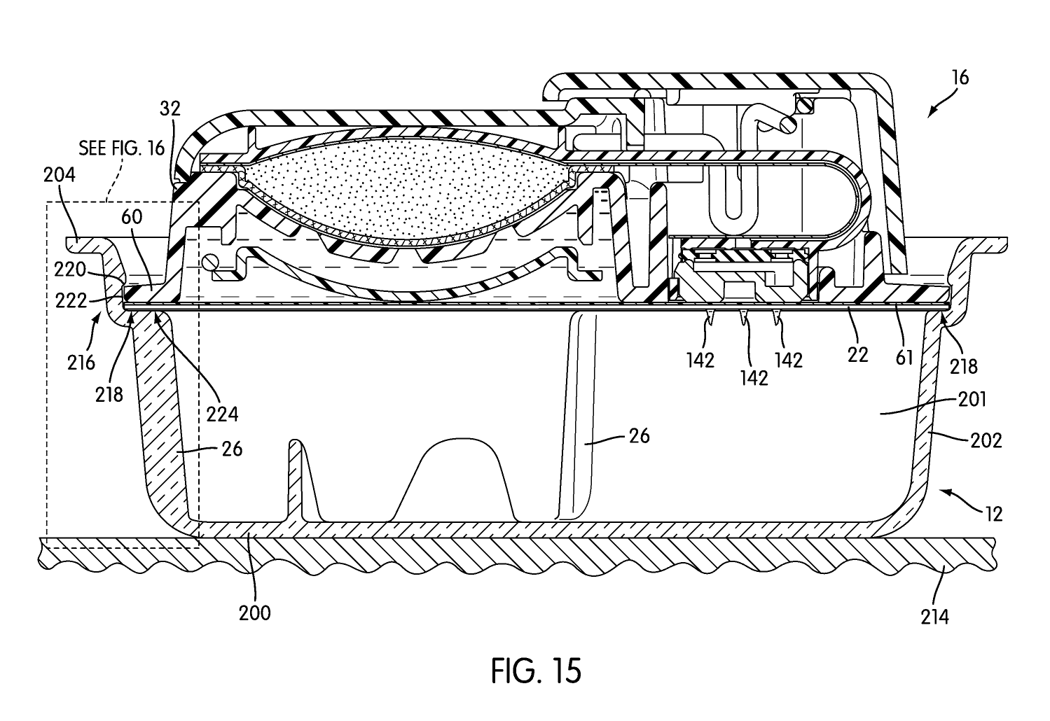

In various embodiments, cover 12 may include a disposal attachment structure to allow cover 12 to function as a sharps-safe disposal container for a drug delivery device, such as drug delivery device 16. Referring to FIG. 15, after cover 12 has been removed from delivery device 16, cover 12 may be placed upside down with top wall 200 placed on a surface 214 (e.g., a table, counter, the ground, etc.). Following delivery of the drug to the subject, drug delivery device 16 is removed from skin 132 and is coupled to the disposal attachment structure of cover 12 such that microneedles 142 are located within chamber 201 of cover 12. With delivery device 16 attached to cover 12 via the disposal attachment structure, cover 12 covers (e.g., conceals, envelopes, houses, etc.) activated microneedles 142 extending below bottom wall 61. With microneedles 142 covered by or located within chamber 201 of cover 12, delivery device 16 and cover 12 may be disposed of without a risk of contact with or potential contamination from microneedles 142.

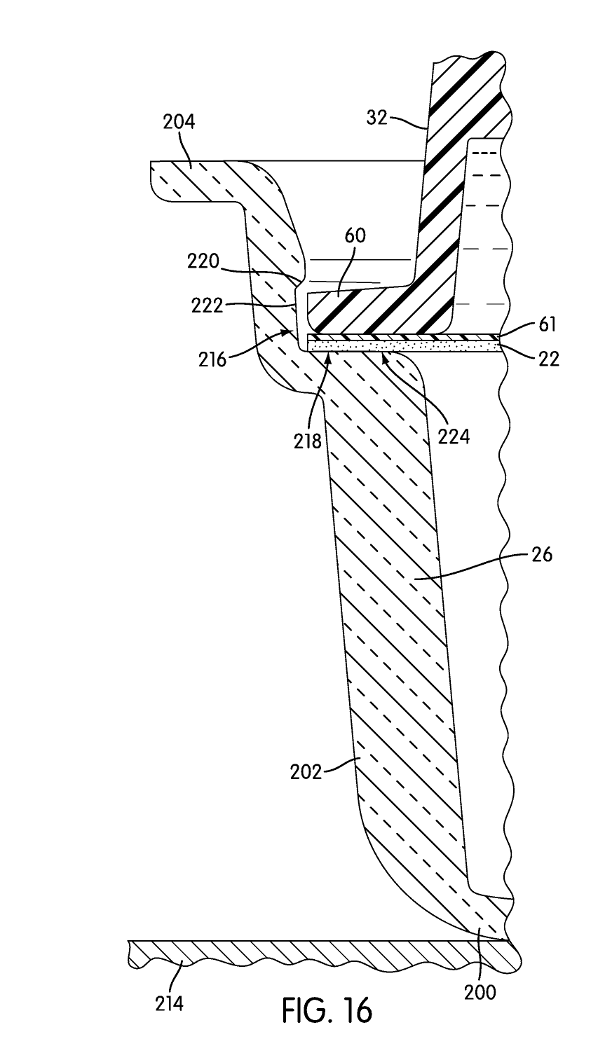

In the embodiments shown in FIGS. 15 and 16, the disposal attachment structure of cover 12 includes a attachment structure 216 and one or more support surfaces 218. Attachment structure 216 includes a bead 220 that extends inwardly from the inner surface of sidewall 202 of cover 12. In one embodiment, bead 220 may be a continuous bead that extends around the inner surface of sidewall 202. In another embodiment, bead 220 may include one or more discreet projections. Positioned below bead 220 is a recess 222 formed in the inner surface of sidewall 202. In this embodiment, delivery device 16 is attached to cover 12 by fitting flange 60 of base portion 32 of delivery device 16 within recess 222 beneath bead 220. Interaction between the surface of bead 220 and the upper surface of flange 60 holds cover 12 to delivery device 16 in the disposal position shown in FIGS. 15 and 16.

In the disposal position of FIGS. 15 and 16, delivery device 16 is supported by one or more support surfaces 218. Support surface 218 extends inwardly from and is generally perpendicular to the inner surface of sidewall 202. In the embodiment shown, support surface 218 is a continuous surface extending from the inner surface of sidewall 202. With top wall 200 in contact with surface 214, support surface 218 generally faces upward as shown in FIG. 15. Support surface 218 engages the portion of adhesive layer 22 generally beneath flange 60. In one embodiment, adhesive layer 22 forms a bond with support surface 218 in the disposal position to help maintain cover 12 and delivery device 16 in the disposal configuration. Further, as shown in FIGS. 15 and 16, generally horizontal surfaces 224 of tabs 26 (shown as facing upward in FIGS. 15 and 16) are contiguous with support surface 218. Thus, in this embodiment, surfaces 224 of tabs 26 also provide support to delivery device 16 in the disposal configuration.

In one embodiment, cover 12 includes a device attachment structure, for example tabs 26, that is a separate and distinct structure or component from the disposal attachment structure of cover 12. For example, surfaces 208 and 210 of tabs 26 which engage the outer surfaces of housing 18 (see FIG. 11) are distinct from bead 220 and recess 222 that engages delivery device 16 in the disposal configuration as shown in FIGS. 15 and 16. In the embodiment shown, the device attachment structure, shown as tabs 26, is located between top wall 200 and the disposal attachment structure, shown as including bead 220 and recess 222, and the disposal attachment structure, shown as including bead 220 and recess 222, is located between the lower edge of cover 12 and the device attachment structure, shown tabs 26. In the embodiment shown in FIGS. 15 and 16, bead 220 is located between flange 204 and recess 222, recess 222 is located between bead 220 and tabs 26, and tabs 26 are located between recess 222 and top wall 200.

Further modifications and alternative embodiments of various aspects of the invention will be apparent to those skilled in the art in view of this description. Accordingly, this description is to be construed as illustrative only. The construction and arrangements of the drug delivery device assembly and the drug delivery device, as shown in the various exemplary embodiments, are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.) without materially departing from the novel teachings and advantages of the subject matter described herein. Some elements shown as integrally formed may be constructed of multiple parts or elements, the position of elements may be reversed or otherwise varied, and the nature or number of discrete elements or positions may be altered or varied. The order or sequence of any process, logical algorithm, or method steps may be varied or re-sequenced according to alternative embodiments. Other substitutions, modifications, changes and omissions may also be made in the design, operating conditions and arrangement of the various exemplary embodiments without departing from the scope of the present invention.

What is claimed is:

1. An apparatus for delivering a drug to a subject, comprising:
a housing;
a microneedle coupled to the housing and configured to extend from the housing when activated;
an activation control coupled to the housing; and
an outer shell comprising:
a top wall having an inner surface;
a sidewall extending from the top wall, the sidewall having an inner surface;
a first attachment structure configured to attach to the housing, wherein the outer shell covers the activation control when the first attachment structure is attached to the housing; and
a second attachment structure configured to attach to the housing, wherein the outer shell covers the activated microneedle when the second attachment structure is attached to the housing.

2. The apparatus of claim 1, wherein the inner surface of the top wall and the inner surface of the sidewall define a central chamber.

3. The apparatus of claim 2, wherein the activation control and the housing are received within the central chamber when the first attachment structure is attached to the housing.

4. The apparatus of claim 2, wherein the activated microneedle is received within the central chamber of the outer shell when the second attachment structure is attached to the housing.

5. The apparatus of claim 1, wherein the first attachment structure includes a tab extending from the inner surface of the sidewall, the tab having an inner surface, the inner surface of the tab engaging the housing to attach the outer shell to the housing.

6. The apparatus of claim 5, wherein the outer shell is configured to be attached to the housing via an interference fit between the tab and the housing.

7. The apparatus of claim 5, wherein the second attachment structure includes a bead extending from the inner surface of the sidewall and a recess formed in the sidewall positioned adjacent to the bead, and further wherein a portion of the housing is received within the recess when the outer shell is attached to the housing via the second attachment structure.

8. The apparatus of claim 7, wherein the portion of the housing is a flange extending from a lower peripheral edge of the housing, and the bead engages the flange to resist movement of the housing relative to the outer shell when the second attachment structure is attached to the housing.

9. The apparatus of claim 7, wherein the tab is located on the sidewall between the recess and the top wall.

10. An apparatus for delivering drug to a subject, comprising:
a housing;
a microneedle configured to extend from the housing when activated; and
an activation control coupled to the housing; and
an outer shell coupled to the housing, comprising:
a top wall having an inner surface;
a sidewall extending from a peripheral edge of the top wall, the sidewall having an inner surface, the inner surfaces of the top wall and the sidewall defining a central chamber;
a first attachment structure coupled to the housing, wherein the housing and the activation control are located within the central chamber when the outer shell is coupled to the housing via the first attachment structure; and
a second attachment structure configured to be coupled to the housing, wherein the activated microneedle is located within the central chamber when the outer shell is coupled to the housing via the second attachment structure.

11. The apparatus of claim 10, wherein the outer shell provides a sharp-safe container for disposing of the microneedle after the drug has been delivered.

12. The apparatus of claim 11, wherein the outer shell is made from a rigid material.

13. The apparatus of claim 10, wherein the housing includes a bottom wall having a lower surface, wherein the lower surface of the housing faces generally toward the top wall of the outer shell when the outer shell is coupled to the housing via the second attachment structure, and further wherein the lower surface of the housing faces generally away from the top wall of the outer shell when the outer shell is coupled to the housing via the first attachment structure.

14. The apparatus of claim 10, wherein the outer shell is coupled to the housing via the first attachment structure prior to activation, and further wherein the outer shell is coupled to the housing via the second attachment structure following drug delivery to facilitate disposal of the microneedle.

15. A method of delivering a drug to the skin of a subject, the method comprising:
providing a microneedle drug delivery device held within a protective cover;
attaching the microneedle drug delivery device to the skin of the subject via an attachment element;
removing the protective cover from the microneedle drug delivery device while the microneedle drug delivery device is attached to the skin of the subject to expose an activation control;
actuating the activation control to trigger insertion of a microneedle into the skin of the subject and to initiate drug delivery via the microneedle;
removing the microneedle drug delivery device from the skin of the subject; and
attaching the microneedle drug delivery device to the protective cover for disposal such that the exposed microneedle is covered by the protective cover.

16. The method of claim 15, wherein the protective cover includes a plurality of tabs configured to be coupled to an outer surface of the microneedle drug delivery device to hold the microneedle drug delivery device within the protective cover.

17. The method of claim 16, wherein the protective cover includes a recess that receives a portion of the microneedle drug delivery device to attach the protective cover to the microneedle drug delivery device.

18. The method of claim 15, wherein the removing the protective cover step includes applying an inwardly directed force to a sidewall of the protective cover.

19. The method of claim 15, further comprising the step of placing the protective cover onto a surface such that a top wall of the protective cover is in contact with the surface.

20. The method of claim 19, wherein the microneedle drug delivery device includes a bottom wall having a lower surface, wherein the lower surface of the bottom wall faces the top wall of the protective cover when the used microneedle drug delivery device is attached to the protective cover.
